(19) United States
(12) Patent Application Publication
(10) Pub. No.: US 2017/0083675 A1
(43) Pub. Date: Mar. 23, 2017

(54) Method for Selecting Patient Subpopulation for Custirsen Treatment

(71) Applicant: OncoGenex Technologies Inc., Vancouver (CA)

(72) Inventors: Cindy Jacobs, Fall City, WA (US); Brent A. Blumenstein, Seattle, WA (US)

(73) Assignee: OncoGenex Technologies Inc., Vancouver (CA)

(21) Appl. No.: 14/984,695

(22) Filed: Dec. 30, 2015

Related U.S. Application Data
(60) Provisional application No. 62/221,488, filed on Sep. 21, 2015.

(51) Int. Cl. G06F 19/00; A61K 31/7088; A61K 31/337; C12N 15/113

(52) U.S. Cl. CPC G06F 19/345; C12N 15/113; A61K 31/7088; A61K 31/337; C12N 2310/111; C12N 2320/31

(57) ABSTRACT

A method of determining if a cancer patient is susceptible to survival prolongation if treatment is augmented with a clusterin-inhibiting pharmaceutical is provided. The method involves measurement of various patient data and a systematic approach to the analysis. A computer-aided system is also provided.

### CROSS-REFERENCE TO RELATED APPLICATIONS

This application claims the benefit of U.S. Provisional Application No. 62/221,488, filed Sep. 21, 2015, which is incorporated herein by reference in its entirety.

### STATEMENT REGARDING FEDERALLY SPONSORED RESEARCH OR DEVELOPMENT (IF APPLICABLE)

None.

### BACKGROUND OF THE INVENTION

The invention relates to the field of oncology medicine, specifically to a more targeted patient-specific treatment for prostate cancer.

Custirsen is a second-generation antisense oligonucleotide (ASO) designed to bind to clusterin (CLU) mRNA, resulting in inhibition of production of human CLU protein. Custirsen has been shown to enhance tumor cell death following treatment with chemotherapy. “Second generation” refers to the chemical modifications that make antisense stable as a pharmaceutical.

There is evidence implicating the presence of CLU within tumor cells and the intensity of its expression with poor prognosis, in patients with esophageal squamous cell carcinoma [1], prostate cancer, [2],[3], breast cancer [4], urothelial cancer, [5], and cervical cancer[6]. Furthermore, preclinical studies leading up to clinical testing showed that CLU knockdown using custirsen could sensitize tumors to chemotherapy and reverses chemotherapy resistance.

An open-label, Phase 1, dose-escalation, safety, pharmacokinetic, and pharmacodynamic study evaluated weekly doses of custirsen in combination with neoadjuvant hormone therapy (NHT) in 25 patients with localized prostate carcinoma prior to radical prostatectomy, and the Phase 2 data for similar test conditions suggested a good outcome in further testing.

Despite the previous findings, in the Phase 3 clinical trial for metastatic castrate-resistant prostate cancer (MCRPC) patients randomized to the custirsen arm versus control (“SYNERGY”), there was no substantial improvement in survival for the experimental group. This result seems to run against all previous findings.

Around the same time as the Synergy trial, another metastatic prostate cancer clinical trial (“STAMPEDE”) assessed abiraterone and enzalutamide in combination with hormone therapy or radiotherapy in combination with hormone therapy. [7]. Survival rates were disappointing, particularly if the patient's performance status was ECOG 1 or 2 compared to 0.

In light of the still unmet need for a satisfactory treatment for metastatic prostate cancer, and in light of the unpredictable nature of the disease in Phase 3 studies, Sponsors for the Phase 3 Synergy trial attempted to understand how to make use of the results of the trial to extend patient survival. Specifically, a means of identifying which prostate cancer patients would be aided by Custirsen therapy was investigated.

### BRIEF SUMMARY OF THE INVENTION

As a consequence of this investigation, it has surprisingly been found that prostate cancer patients with the poorest prognoses, as determined using indicators not specifically related to clusterin expression receive the greatest benefit in terms of prolongation of life when clusterin expression is reduced as part of a therapeutic regimen, for example through the administration of custirsen.

Thus, in a first aspect, the present invention provides a method for determining if a cancer-afflicted patient is susceptible to survival prolongation through treatment with a clusterin-inhibiting agent in combination with a chemotherapy agent, the method comprising the steps of:

(a) obtaining a plurality of test results for a patient, said test results being selected from the group consisting of:

(i) a Performance Scale result,

(ii) the presence of metastatic lesions on the patient's liver,

(iii) blood prostate specific antigen (PSA) level,

(iv) blood lactose dehydrogenase (LDH) level, and

(v) hemoglobin (Hb) levels;

(b) comparing each of the obtained test results to a predetermined value indicative of a poor prognosis; and
(c) if any two of the resulting five measurements indicate poor prognosis in a cancer patient concluding that said patient is susceptible to survival prolongation through treatment with a clusterin-inhibiting agent in combination with a chemotherapy agent.

In some embodiments, a combination of any three of more, four or more, or all five of the measurements indicative of a poor prognosis is used as a basis for the conclusion that a patient is susceptible to survival prolongation.

A further aspect of the invention comprises determining if a cancer-afflicted patient is susceptible to survival prolongation through treatment with a clusterin-inhibiting agent in combination with a chemotherapy agent, and then treating the patient with a clusterin-inhibiting agent in combination with chemotherapy.

In preferred embodiments, the clusterin-inhibiting agent is custirsen, and the chemotherapy treatments includes treatment with a taxane, such as docetaxel.

The method of the invention may be practised using a dedicated system which performs the comparison of input data to stored information concerning the predetermined values indicative of a poor prognosis and outputs a result.

### BRIEF DESCRIPTION OF THE SEVERAL VIEWS OF THE DRAWING

In drawings which illustrate embodiments of the invention,

FIG. 1 is a graphical representation of survival differences for control versus experimental arms for all study patients in the SYNERGY trial;

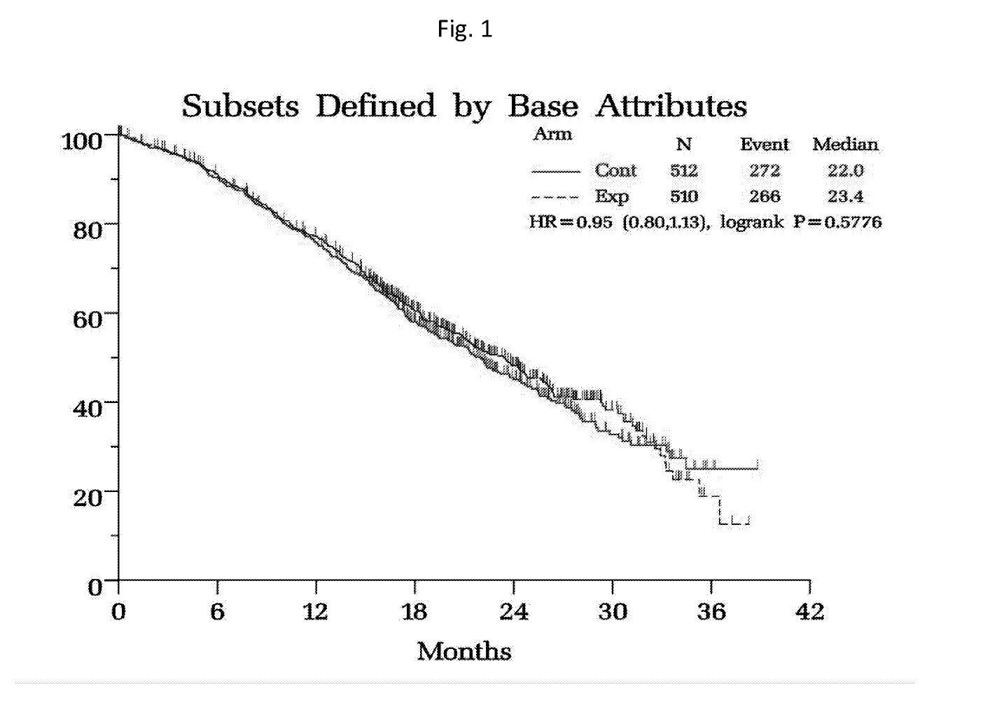

FIG. 2 is a graphical representation of survival differences between “Good vs Poor Prognosis” patients as determined by to the presence of liver metastases for those patients;

FIG. 3 is a graphical representation of survival differences, split into Good vs. Poor prognosis patients categorized according to their Karnofsky Performance Status in the Synergy trial;

FIG. 4 is a graphical representation of Survival differences split into Good vs. Poor prognosis patient categories according to (high) levels of prostate-specific antigen (PSA) in the Synergy trial;

FIG. 5 is a graphical representation of survival in control versus experimental arms split into good vs. poor prognosis patient categories as classified by patient levels of LDH, AP and Hemoglobin in the Synergy trial;

FIG. 6 is a graphical representation of survival differences showing control versus experimental arms when classified as good vs. poor patient prognosis according to a combined assessment for various prognostic factors, termed “SCAPE2”; and

FIG. 7 is a table showing survival differences for good and poor prognosis patients within control versus experimental arms when classified according to SCAPE2 results in the Synergy trial.

### DETAILED DESCRIPTION OF THE INVENTION

A method for selecting patients who will benefit from the addition of Custirsen to their cancer treatment is provided. Identification of such patients, and the subsequent addition of custirsen to the treatment regimen provides the opportunity to increase the chance that the patients having advanced prostate cancer will experience increased survival times. It will be appreciated that concepts of increased survival times are statistical in nature, and that it is impossible to know what the actual survival time for any individual patient would have been had been had a different treatment regimen been employed. Nevertheless, the present invention provides an opportunity to identify a treatment regimen that is statistically likely to provide increased survival times for a given patient.

The method according to the invention was motivated by, and engineered using, data derived from the Phase 3 “SYNERGY” trial (corporate name OGX-011-11). This was a randomized, open-label Phase III study comparing custirsen in combination with standard first-line docetaxel/prednisone to docetaxel/prednisone alone in men with metastatic castrate resistant prostate cancer (mCRPC). The primary study endpoint was the comparison of survival time distribution for patients randomized to the custirsen arm versus the control arm.

Top-line survival results released to the public on Apr. 28, 2014 indicated that the addition of custirsen to standard first-line docetaxel/prednisone therapy did not meet the primary endpoint of a statistically significant improvement in overall survival in men with metastatic castrate-resistant prostate cancer (CRPC), compared to docetaxel/prednisone alone (median survival 23.4 months vs 22.2 months, respectively; hazard ratio 0.93 and one-sided p value 0.207).

A previous study, the 82 patient Phase 2 (OGX-011-03) study of Custirsen in metastatic castrate-resistant prostate cancer (mCRPC) with similar study arms (randomly receiving either custirsen in combination with docetaxel/prednisone or docetaxel/prednisone alone) showed great promise. The median overall survival in that study was 23.8 months for the custirsen/docetaxel/prednisone arm and 16.9 months for the docetaxel/prednisone arm, a difference of 6.9 months in favour of the custirsen arm (unadjusted HR=0.606; 95% CI 0.36-1.02).

After the results of the Phase 3 were announced, additional exploratory data analyses revealed that in the Phase 2 study, 50% of patients had an overall poorer performance status, compared to 30.8% in the Phase 3 Synergy trial. This indicated that potentially the sickest patients had the most benefit from custirsen.

In retrospective analyses, a prognostic scoring system has been developed in the control arm using multiple variable modelling and was used to dichotomize patients into good and poor prognosis. The analysis included complete data for 984 patients. The patients were retrospectively divided into “poor” prognosis and “good” prognosis. Median survival for the poor and good prognosis groups in the control arm was 14.0 months (m) and 30.4 m, respectively (HR=3.66).

The Custirsen HR effect differed between poor and good prognosis groups (interaction P=0.069). The HR estimate for Custirsen survival benefit for those in the poor prognosis group was 0.73 (95% CI: 0.59 to 0.90) and 1.02 (95% CI: 0.76 to 1.37) for those in the good prognosis. When analyzed separately (n=492), the median survival in the poor prognostic group was 17.0 m in the custirsen arm vs. 14.0 m in the control arm (HR=0.73, 95% CI: 0.59 to 0.90, P=0.004).

These findings were then used to develop the method that a medical practitioner could use to screen patients for Custirsen therapy. Originally, seven factors were considered. However we found that the same accuracy could be accomplished with five factors, and even then, if only two were within the desired “Poor prognosis” range, the patient's survival would be lengthened by the addition of Custirsen to their treatment.

Specifically, the results showed that over 40% of men in the trial had at least two of five risk factors for poor prognosis that were analyzed above and also common prognostic factors known for shorter prostate cancer survival outcomes. In these men, the more simplified prognostic analysis also showed a 27% lower risk of death when Custirsen was used in combination with first-line docetaxel compared to docetaxel alone.

### DEFINITIONS

“ASO” is an acronym for “Antisense Oligonucleotide”. Antisense therapeutics are based on the premise that sequences of single-stranded nucleic acids (antisense oligonucleotides or ASOs) will bind to complementary strands of nucleic acids through hybridization. A cancer cell with overexpression of a specific protein produces an abundance of messenger RNA that is translated into excess protein[8]. The introduction of a specific complementary or “antisense” strand of single-stranded DNA can bind to the abundant mRNA strands, leading to degradation before translation can occur and reduction in protein levels of the target gene[9].

Various antisense chemistries have been evaluated to generate potential drug candidates for cancer therapy. Over the last ten years, considerable effort has been made by numerous groups to improve the in vivo potency of ASOs by modifications of the phosphodiester-linkage and heterocyclic structure of the sugar. Advances in modified nucleic acid chemistry[10-12] have yielded “second-generation” ASO modifications which improve both RNA binding affinity and resistance to nuclease degradation, thereby, increasing its half-life and resulting in increased potency.

“Clusterin-inhibiting agents” are agents that when administered to a patient result in a reduction in the amount of clusterin. Clusterin-inhibiting agents may be oligonucleotides that inhibit expression of clusterin by a sequence specific interaction with DNA or RNA in cells of the patients. In specific embodiments, the clusterin-inhibiting agent is an ASO.

“Custirsen” is the USAN name for a clusterin-inhibiting compound, specifically a clusterin-specific antisense described in U.S. Pat. No. 6,900,187 or CAS Registry No. 685922-56-9, WHO number 9012. Another corporate name for custirsen was OGX-011. Custirsen has been supplied for clinical trials as a 160 mg per 8 mL solution resulting in a 20 mg/mL concentration for custirsen in a 10 mL glass vial. Each patient received 640 mg total via continuous intravenous infusion.

Second-generation chemistry, used in custirsen sodium, applies 2′-O-(2-methoxyethyl) (2′-MOE modification) at the 2 position of the carbohydrate moiety on both ends of the oligonucleotide, resulting in increased target binding affinity, resistance to degradation, and substantially better tissue pharmacokinetics. The improved affinity of a second-generation drug is attributable to its design and composition. In particular, second-generation drugs are composed of both RNA-like and DNA-like nucleotides, while first-generation drugs are entirely DNA-like. Because RNA hybridizes more tightly to RNA than to DNA, the second-generation drugs have a greater affinity for RNA targets and therefore greater potency and in significantly improved tissue half-life in vivo.[13] This produces a longer duration of action, allowing less frequent dosing. Finally, 2′-MOE ASOs have been reported to have a more attractive safety profile than unmodified phosphorothioate ASOs.

“Custirsen-like compounds” are clusterin-inhibiting compounds including antisense and siRNA, antibodies or fragments thereof, or peptides, all of which inhibit clusterin expression, or which reduce levels of active clusterin by direct inhibition of clusterin protein. These custirsen-like compounds include clusterin-specific ASO, double stranded RNA, and clusterin-targeting antibodies and peptides;

“HR” is an acronym for “Cox regression Hazard Ratio”. Hazard ratio is ratio of overall survival hazard rates for first category over second category. Values less than one indicate lower risk of death for first category relative to second category.

“HRPC” is an acronym for “Hormone-Refractory Prostate Cancer”, “mCRPC” is an acronym for “Metastatic Castrate Resistant Prostate Cancer”, and “AIPC” is an acronym for “Androgen-Independent Prostate Cancer”. These are related terms, but the one used in the application is mCRPC as it is the identifier used in the clinical trials. Many prostate cancers that initially respond to hormone eventually stop responding to this treatment. This is referred to as castration-resistant prostate cancer. Castration-resistant prostate cancers need much lower levels of androgen to grow than androgen-sensitive cancers.

“NHT” is an acronym for “Neoadjuvant Hormone Therapy”. There are different forms to suppress androgens which may be causing prostate cancer to grow. Antiandrogens that are approved to treat prostate cancer include flutamide, enzalutamide, bicalutamide, and nilutamide. Antiandrogens are oral medications. Androgen synthesis inhibitors, such as ketoconazole, aminoglutethamide, and abiraterone acetate, may also be used. (www.cancer.gov)

The Phase 2 study entitled: “A Randomized Phase II Study of OGX-011 in Combination with Docetaxel and Prednisone or Docetaxel and Prednisone Alone in Patients with Metastatic Castration Resistant Prostate Cancer” consisted of 80 patients and measured the rate of PSA decline 50% as a primary endpoint, and tolerability, objective response rate, PFS and overall survival as secondary endpoints. The results were published in 2009.[14]

The SYNERGY Trial refers to Protocol OGX-011-11, “A Randomized Phase 3 Study Comparing Standard First-Line Docetaxel/Prednisone to Docetaxel/Prednisone in Combination with Custirsen (OGX-011) in Men with Metastatic Castrate Resistant Prostate Cancer”. The results were discussed in an Apr. 28, 2014 Press Release entitled: OncoGenex Announces Top-Line Survival Results of Phase 3 SYNERGY Trial Evaluating Custirsen for Metastatic Castrate-Resistant Prostate Cancer.

The AFFINITY Trial refers to Protocol Protocol OGX-011-12, “A Randomized Phase 3 Study Comparing Cabazitaxel/Prednisone in Combination with Custirsen (OGX-011) to Cabazitaxel/Prednisone for Second-Line Chemotherapy in Men with Metastatic Castrate Resistant Prostate Cancer”.

“OS” stands for “Prolonged Overall Survival”.

The phrase “obtaining a plurality of test results” encompasses both performance of steps to obtain the test results as part of the method of the invention, and also, obtaining the results of previously conducted tests, for example from a patient's medical records, and combinations thereof. It will be appreciated that the specific tests of this application are commonly performed on cancer patients, and that the method of the invention may therefore be practiced without requiring a repeat of some or all of the tests.

The term “Performance Scale result” refers to the result of an evaluation that provides an indication of the patient's functional capabilities. Two common performance scales are the Karnofsky scale and the ECOG scale. As shown in the following Table 1, these two Performance Scales use somewhat different language to describe the functional state of a patient, and inverse numerical indicators.

TABLE 1

| | Karnofsky | ECOG | |
|---|---|---|---|
| Karnofsky Status | Grade | Grade | ECOG Status |
| Normal, no complaints | 100 | 0 | Fully active, able to carry on all pre- |
| | | | disease performance without |
| | | | restriction |
| Able to carry on normal | 90 | 1 | Restricted in physically strenuous |
| activities. Minor signs or | | | activity but ambulatory and able to |
| symptoms of disease | | | carry out work of a light or sedentary |
| | | | nature, e.g., light house work, office |
| | | | work |
| Normal activity with effort | 80 | 1 | Restricted in physically strenuous |
| | | | activity but ambulatory and able to |
| | | | carry out work of a light or sedentary |
| | | | nature, e.g., light house work, office |
| | | | work |
| Care for self. Unable to | 70 | 2 | Ambulatory and capable of all selfcare |
| carry on normal activity or | | | but unable to carry out any work |
| to do active work | | | activities. Up and about more than |
| | | | 50% of waking hours |
| Requires occasional | 60 | 2 | Ambulatory and capable of all selfcare |
| assistance, but able to care | | | but unable to carry out any work |
| for most of his needs | | | activities. Up and about more than |
| | | | 50% of waking hours |
| Requires considerable | 50 | 3 | Capable of only limited selfcare, |
| assistance and frequent | | | confined to bed or chair more than |
| medical care | | | 50% of waking hours |
| Disabled. Requires special | 40 | 3 | Capable of only limited selfcare, |
| care and assistance | | | confined to bed or chair more than |
| | | | 50% of waking hours |
| Severly disabled. | 30 | 4 | Completely disabled. Cannot carry on |
| Hospitalisation indicated | | | any selfcare. Totally confined to bed |
| though death nonimminent | | | or chair |
| Very sick. Hospitalisation | 20 | 4 | Completely disabled. Cannot carry on |
| necessary. Active | | | any selfcare. Totally confined to bed |
| supportive treatment | | | or chair |
| necessary | | | |
| Moribund | 10 | 4 | Completely disabled. Cannot carry on |
| | | | any selfcare. Totally confined to bed |
| | | | or chair |
| Dead | 0 | 5 | Dead |

Measurements of patient baseline levels of requisite variables was performed in the Synergy trial. Prostate-Specific Antigen or PSA is a protein measured in the blood. Levels of 4.0 ng/mL and less are considered normal, with anything above that level of protein in the blood being considered elevated. PSA is usually part of a panel of tests and examinations rather than a stand-alone test, because of the possibility of benign elevation.

Opioid use includes types and amounts of opioids prescribed to patients. These can be oral or intravenous, outpatient or in-hospital. “Opioid use” as a patient characteristic can be used as an indicator of pain and subsequently, illness.

Lactic Acid Dehydrogenase or Lactate Dehydrogenase (LDH) is an enzyme that is native to the human body. Levels of LDH increase in response to cell damage. LDH levels are tested routinely in blood and normal levels vary from person to person and lab to lab, but range from 140-280 Units per litre of blood (WebMd online Medical Reference, Healthwise Incorporated).

Alkaline Phosphatase or ALP is a universally-present blood enzyme. Elevated levels in adults are over 100 Units per litre (WebMd online Medical Reference, Healthwise Incorporated).

Haemoglobin (Hb) is a protein that is an indicator of healthy blood. Below normal for males is anything under 140 g/L, and in females is under 123 g/L. Haemoglobin levels can also be described as g/dL, or one tenth of the SI Units used here.

The term “predetermined value indicative of poor prognosis” refers to a value for each of the tests identified herein that has been found to the inventors to provide an appropriate indication of susceptibility of a patient to survival prolongation if a clusterin-inhibiting agent such as Custirsen is added to their treatment. Specific predetermined values for each of the tests are shown in Table 2.

TABLE 2
Final “SCAPE2 systematic patient identification tool”

| | | |
|---|---|---|
| 1. | Karnofsky Performance Status | ≦80 |
| 2. | Liver metastasis | ≧1 |
| 3. | LDH (dichotomized) - | LDH ≧360 IU/L |
| 4. | PSA (dichotomized) - | ≧150 ng/mL |
| 5. | Hemoglobin (dichotomized) | <120 g/L |

Chemotherapy agents for cancer includes traditional cytotoxic chemotherapeutic agents such as alkylating agents, anti-metabolites, and cytotoxic agents. Commonly used chemotherapeutic regimes are cyclophosphamide, methotrexate, 5-fluorouracil (CMF) and doxorubicin, cyclophosphamide (AC) which are selected for breast cancer treatment; methotrexate, vincristine, doxorubicin, and cisplatin (MVAC) for bladder cancer, cyclophosphamide, doxorubicin, vincristine, (CAV) for lung cancer, 5-fluorouracil, folinic acid, oxaliplatin (FOLFOX) for colorectal cancer, and docetaxel and prednisone for prostate cancer. Taxane agents indicated for hormone refractory or androgen-independent prostate cancer include paclitaxel (Taxol™) and docetaxel (Taxotere™) and cabazitaxel (Jevtana™). Abiraterone (Zytiga™) and enzalutamide (Xtandi™) are newer chemical/biological therapies for prostate cancer that may also be used in combination with custirsen.

Docetaxel is a semisynthetic analog of paclitaxel using a precursor extracted from the needles of the European yew tree. Docetaxel's high affinity for binding to microtubules enhances tubular polymerization, leading to inhibition of mitosis and cell division. Docetaxel is a cell-cycle specific agent with activity in the mitotic phase.

There are a number of strategies in the administration of chemotherapeutic drugs used today, and any of these consistent with the drug being administered may be employed.

### EFFICACY OF THE INVENTION

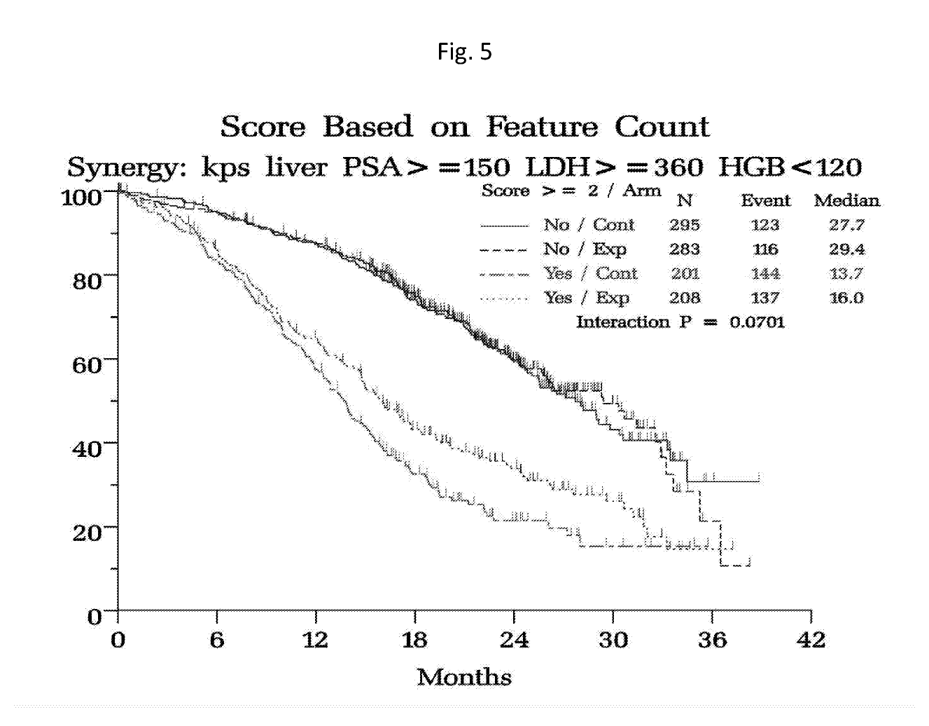

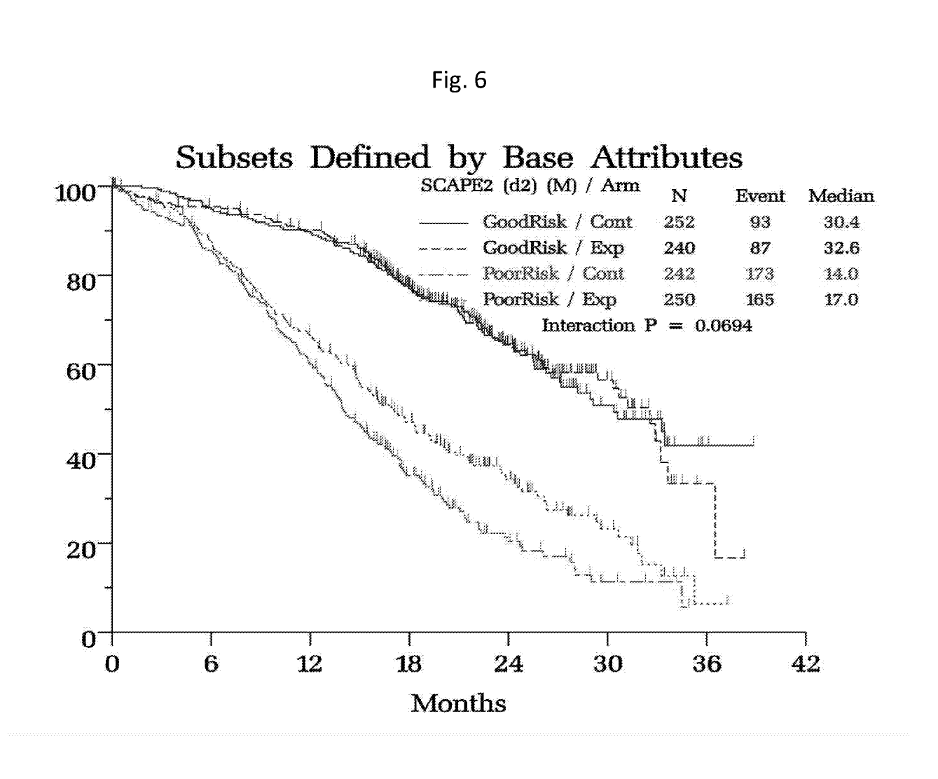

FIGS. 5 and 6 demonstrate the efficacy of the invention for identifying patients that benefit in terms of prolonged survival from the addition of a clusterin-inhibiting agent, namely Custirsen, to their treatment regimen.

FIG. 5 shows the patient results from the Synergy Trial, when patients are classified retrospectively into four groups. The top two lines represent outcomes for patients that did not have a poor prognosis indicated by all five criteria of the SCAPE2 model. There is no discernible difference in survival between patients in this group to whom Custirsen was administered, and those to whom it was not. The bottom line represents survival for patients determined to exhibit all five indicators of poor prognosis to whom Custirsen was not administered. The next line up represents survival for patients determined to exhibit all five indicators of poor prognosis to whom Custirsen was administered. While these poor prognosis patients had shorter survival times than either group of good prognosis patients, the survival times for poor prognosis patients who received Custirsen treatment is clearly prolonged.

FIG. 6 shows results for a similar evaluation when classification as poor risk was made if any two of the five indicators in the Scape2 model met the poor prognosis parameters. The prolongation of life for poor risk patients is observed using only two of the indicators as well.

Methods

### PSA, Exploratory Biomarker, and Custirsen PK Specimens

Specimen collection kits (containing all required blood collection tubes, storage vials and laboratory order form) were provided to each of the participating clinical sites. PSA, Clusterin and Other Exploratory Biomarker Testing

Blood (8 mL) was collected at the clinical site. For serum samples, serum after clotting and centrifugation, was separated into six storage vials and frozen at −80° C. at the clinical site (Note: short-term storage at −20° C. was acceptable). Serum and plasma samples were batched and shipped frozen to the central laboratory. The remaining vials remained frozen at the central laboratory at −80° C. and were shipped to one or more third party laboratory(s) for biomarker testing.

PSA samples were analyzed by a central lab, and the results were communicated to the sites. Serum clusterin and other exploratory biomarker testing were batch tested and results sent directly to the Sponsor of the study, when appropriate. Biomarker results were not made available to the sites while the trial was ongoing. Blood samples were collected from patients per the Procedure Schedule in Section 6.1 and further detailed in Section 6.2 of the protocol.

### Custirsen PK Levels

Plasma (2 mL K3 EDTA tube) were collected at the clinical site. After centrifugation, plasma were separated into a storage vial and frozen at −80° C. before forwarding to the central laboratory for testing.

### CTC Enumeration

Blood (7.5 mL) was collected in CellSave™ tubes at selected sites. The tube were immediately inverted 8 times to mix with the EDTA and preservative fluid, and then shipped by overnight courier to one of the central laboratory. Samples/tubes were stored and shipped at room temperature (15-30° C.)

Metastases in the liver are assessed by various radiography (whole body) or computed tomography methods. CT scans should be performed with cuts of 10 mm or less in contiguous slice thickness. Spiral CT should be performed using a 5-8 mm contiguous reconstruction algorithm. Magnetic resonance imaging (MRI) techniques for liver have been studied and methods are well known.[16]

### Example 1

### The Synergy Study

SYNERGY (OGX-011-11) was a randomized, open-label study in about 1000 patients comparing custirsen in combination with standard first-line docetaxel/prednisone to docetaxel/prednisone alone in men with metastatic castrate resistant prostate cancer (mCRPC). The primary study endpoint was the comparison of survival time distribution for patients randomized to the custirsen arm versus the control arm.

There have been 1,022 men enrolled into SYNERGY at 148 cancer centers throughout North America, Europe, Israel and South Korea. SYNERGY completed enrolment in 2012 and final survival results were announced in mid-2014. In the investigational arm of the trial, custirsen was administered as a weekly infusion of 640 mg following three loading doses, in combination with docetaxel and prednisone given as 3-week cycles. Patients in the comparator arm received docetaxel and prednisone without custirsen. In both arms, patients were treated until disease progression, unacceptable toxicity, or completion of up to 10 cycles, unless additional cycles were deemed beneficial by the treating physician.

Custirsen received Fast Track designation from the FDA for the treatment of progressive metastatic prostate cancer in combination with docetaxel/prednisone.

### Treatment

For Arm A patients only, three separate administrations of custirsen were given during Day −9 to Day −1 (Loading Dose Period) prior to beginning Day 1 of Cycle 1.

Following completion of the loading dose period, 640 mg custirsen was given IV weekly on Days 1, 8, and 15 of each 21 day cycle. Custirsen was administered prior to 75 mg/M2 IV of docetaxel on Day 1 of each 21 day cycle.

For the Arm B (no Custirsen) Patients, the first doses of docetaxel and prednisone were administered within 4 days following randomization.

Both Arm A and Arm B Patients:

Docetaxel (75 mg/M2) was administered IV on Day 1 of each 21 day cycle.

Oral prednisone (5 mg twice daily for a total of 10 mg/day) will begin on Day 1 of Cycle 1 and will continue, at a minimum, through the completion of the final treatment cycle. Patients who cannot tolerate prednisone will be eligible for the study. The reason for intolerance must be documented on the case report form (CRF). If a patient is receiving more than the planned dose of 10 mg of prednisone per day (or steroid equivalent) at screening, the dose should be reduced to 10 mg of prednisone per day and maintained for at least 7 days prior to randomization.

Treatment cycles were continued until disease progression, unacceptable toxicity, or completion of 10 cycles.

Blood was collected including 5 mL anticoagulated blood for hematology including tests for WBC, haemoglobin, absolute neutrophils and lymphocytes, platelet count; and additional 5 mL blood for serum chemistries including:

Albumin, serum creatinine, SGOT (AST), SGPT (ALT), bilirubin (total), electrolytes (Na, K), calcium (total) and phosphorus, alkaline phosphatase, LDH; an additional 15.5 mL total blood for serum clusterin, PSA and other exploratory biomarkers (8 mL) and for selected sites additional CTC assessment (7.5 mL).

Blood for CTC assessment was collected in CellSave™ tubes and shipped by overnight courier to the designated central laboratory centers.

Top-line survival results indicate that the addition of custirsen to standard first-line docetaxel/prednisone therapy did not meet the primary endpoint of a statistically significant improvement in overall survival in men with metastatic castrate-resistant prostate cancer (CRPC), compared to docetaxel/prednisone alone (median survival 23.4 months vs 22.2 months, respectively; hazard ratio 0.93 and one-sided p value 0.207.[17, 18]

### Example 2

### Significant Reduction in Risk of Death Observed in Those Patients with Most Common Risk Factors that Drive Poor Outcomes in Prostate Cancer

To understand the less favourable results of the SYNERGY trial as compared to its predecessor Phase 2 trial, a reanalysis of existing data was undertaken.

Multivariable statistical modelling (MSM) was performed to explore the relationship between putative prognostic baseline attributes and survival outcome using only the synergy control arm data, thereby allowing identification of models of prognosis without the influence of custirsen treatment. The variables selected for exploration in the MSM were chosen based on previously published prognostic modelling of completed Phase 3 prostate cancer trials

A retroactive analyses of patients in the SYNERGY trial examined the variables listed below:

Presence of metastases including visceral metastasis and liver metastasis,

Karnofsky Performance status at baseline,

opioid use for prostate cancer,

radiographic progression,

and baseline Laboratory values for Prostate-Specific Antigen (PSA), Alkaline Phosphatase (ALP), Lactic Acid Dehydrogenase (LDH), Hemoglobin (Hb), Neutrophils, Platelets, and Lymphocytes.

Based on the Synergy control arm data, MSM were fit using proportional hazard regression (also known as Cox regression) with survival as the outcome. The variable selection methods included hierarchical step-down variable section. Up to third order interactions between the variables were assessed and continuous variables were dichotomized by their overall median value. Models that included the higher order interactions fit very well and were successfully prognostic but were based on “over-fitting” the data. A simpler model based on balancing between over-fitting and parsimony was the guiding principle (“overfitting” refers to the situation in which a statistical model describes noise instead of the true relationship. It can occur when a model has a lot of parameters and not enough data points).

The scores on these seven indicators were then used to classify patients into “good” and “poor” prognostic subsets based on medians test results and in the context of our medical judgement. When patients in the control arm (n=494) were thus divided, median survival differed markedly between the subgroups in favour of the good prognosis subgroup (30.4 months for the good prognosis subgroup and 14.0 months for the poor prognosis subgroup).

Further analyses evaluated the score derived from the control arm in the whole set of patients in SYNERGY. Complete baseline data were available for 984 of 1022 total patients (94%), with n=490 in the custirsen arm.

Survival benefit for custirsen treatment was observed in the poor prognosis subgroup. The hazard ratio (HR) estimate for survival among poor prognosis patients showed a better survival outcome in the custirsen arm than in the control arm (interaction P=0.069), as shown in FIG. 6. The HR estimate for custirsen survival benefit was 0.73 (95% CI: 0.589 to 0.902) for patients in the poor prognosis subgroup and 1.02 (95% CI: 0.760 to 1.37) for patients in the good prognosis subgroup. An HR close to 1.0 means equal survival between the treatment groups.

Also shown in FIG. 6, the median survival for patients in the custirsen arm was 17.0 months for the poor prognosis subset and 32.6 months for the good prognosis subset.

### Example 3

### Designing the SCAPE2 Systematic Patient Identification Tool

In order to identify the subset of factors that best predicted overall survival, all seven factors discussed above were used in a Cox proportional hazards multiple regression model with stepwise variable selection and a significance level of 0.05 for variables to enter and remain in the model. We called the following list of prognostic readouts “SCAPE”.

Karnofsky performance status, liver metastases, opioid use, LDH levels, haemoglobin levels, PSA levels, and alkaline phosphatase levels.

The variables in the final model were baseline performance status, visceral disease status, and treatment arm. The p-values associated with each of these factors were less than 0.05. The analyses demonstrated a lower risk of death for subjects on treatment Arm A (Custirsen) after adjusting for the effect of performance status and visceral disease status. The analyses of effect of treatment arm on overall survival according to the number of cycles received demonstrated that the treatment effect on survival appeared to consistently favor Arm A regardless of the number of cycles received. The hazard ratio (HR) for comparing the treatment groups (custirsen+docetaxel vs docetaxel alone) for the 22 subjects who received five or fewer cycles (HR=0.78) was similar to the HR for the 36 subjects who received all 10 cycles (0.69); the HR for the 24 subjects who received between six and nine cycles was 0.15 (An HR value of less than one is favourable).

To create a systematic tool that was not susceptible to differences in patient values due to non-cancer conditions, and simple enough for medical practitioners to quickly assess their patients for treatment options, the SCAPE criteria were examined for ease of use as well as quality of information. Finally, it was determined that if any two of the following 7 criteria were “positive” or indicated poor prognosis, the patient would benefit from Custirsen treatment.

1. Karnofsky ≦80

2. Positive for Liver metastasis

3. Positive for Opioid use for prostate cancer (stratifier)

4. Elevated LDH (dichotomized)

5. Below normal Hemoglobin (dichotomized)

6. Elevated PSA (dichotomized)

7. Elevated Alkaline phosphatase (dichotomized).

From the seven significant prognostic factors (SCAPE) defined in the multivariable analysis of SYNERGY, SCAPE2 was refined into a more simplified 5-criteria characterization. The proposed 5 criteria include Karnofsky performance status, the presence of even one liver metastasis, below normal hemoglobin levels, above normal LDH level and PSA level as defined below.

Opioid use for prostate cancer pain and alkaline phosphatase levels were dropped because it was decided that opioid use specific for cancer pain versus other indications can be difficult for patients to report (subjective), accurately defining opioid-containing medications on a country-by-country basis could interject error, and individual countries can prescribe opioid-use quite differently. Furthermore, it was determined that concomitant medications, such as various bone-targeted agents, and presence of liver or bone metastasis could confound alkaline phosphatase results.

Therefore the subpopulation of prostate cancer patients who would be susceptible to prolonged survival when treated with custirsen is defined as patients who satisfy 2 or more of the following 5 morbidity parameters:
a) Poor performance status, defined by Karnofsky performance status ≦80%;
b) Presence of liver metastasis;
c) Presence of anemia, defined by a hemoglobin <120 g/l (Note: At this level, RBC transfusions for patients with anemia are not likely to obtain levels at or higher than 120 g/l so anemia will still be identified using this criteria);
d) High Lactic Acid Dehydrogenase (LDH), defined by a LDH 360 IU/l (Note: The 360 IU/l level is the median LDH value for the poor prognostic subgroup in the SYNERGY trial. The upper limit of normal for LDH testing at each site was considered, but the extreme variability within the various countries and sites for upper limit of normal precluded this as a standardized option [upper limit values ranged 112 IU/l-620 IU/l]); and
e) High serum prostate specific antigen (PSA) level, defined by a PSA 150 ng/ml (Note: The 150 ng/ml level is based on the median PSA value of the poor prognostic subgroup in the SYNERGY trial and is in a similar range identified by Halabi et al as a prognostic variable).

Specifically, the SYNERGY between-arm hazard ratio in the subset of poor prognosis patients classified using the seven criteria spread was estimated as 0.728, and the between-arm hazard ratio estimate was 0.729 for those classified as poor prognosis using just the five prognostic factors defined above. This was not considered a substantial difference.

Thus, SCAPE2 (d2) systematic patient identification tool was arrived at for identifying the patients whose survival would be improved with custirsen as part of their treatment. In particular, this modeling demonstrated the SCAPE2(d2) power to identify a group of poor prognosis patients particularly responsive to custirsen treatment for use in future studies and eventually patient treatment. SCAPE 2(d2) will assist first-line or second-line chemotherapy patient identification.

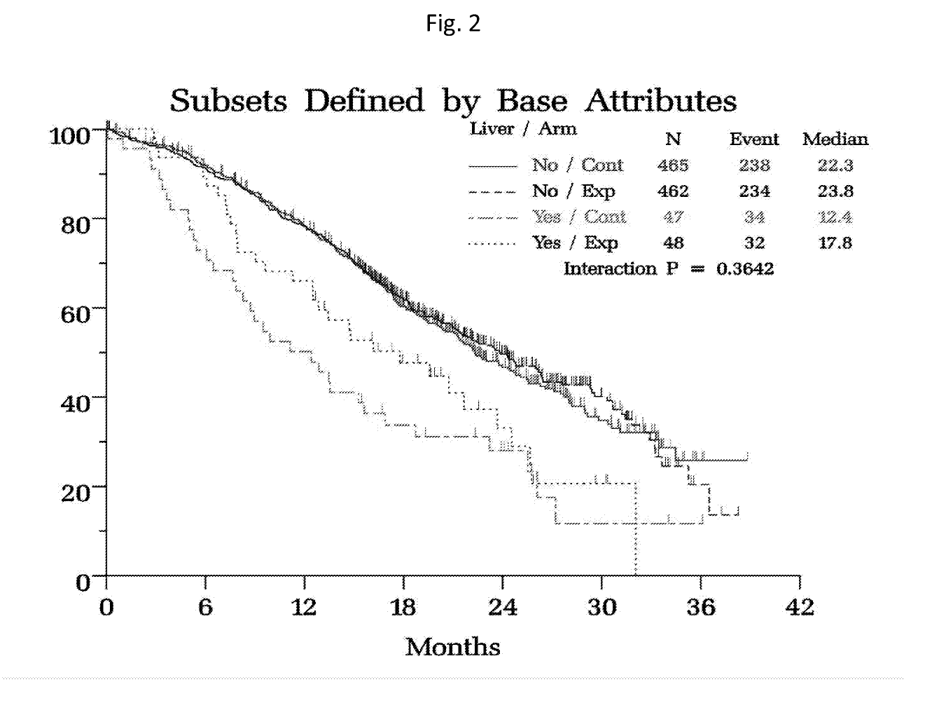

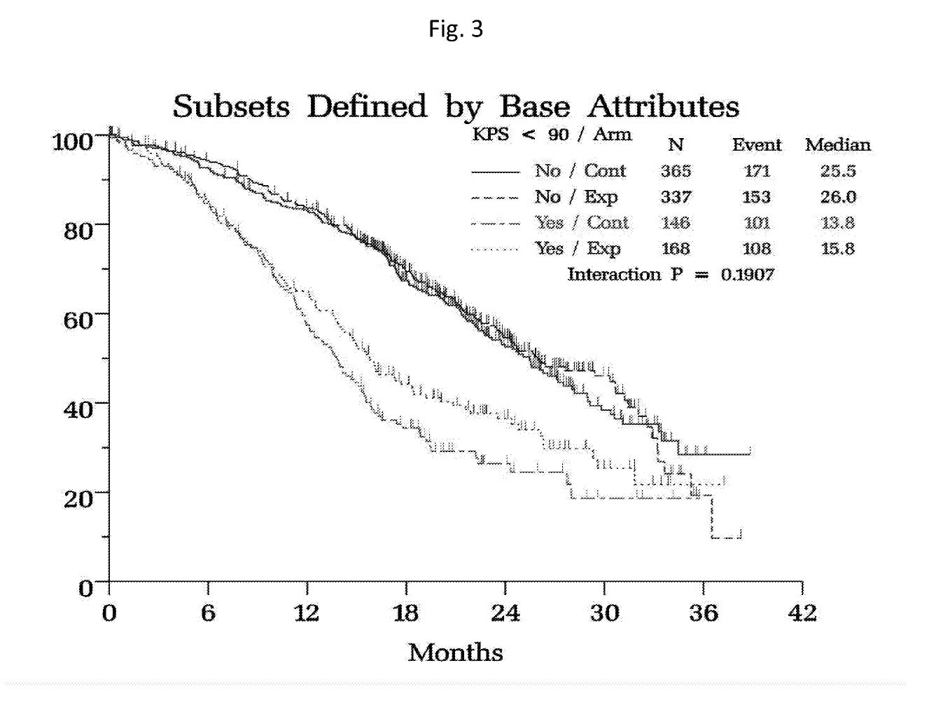

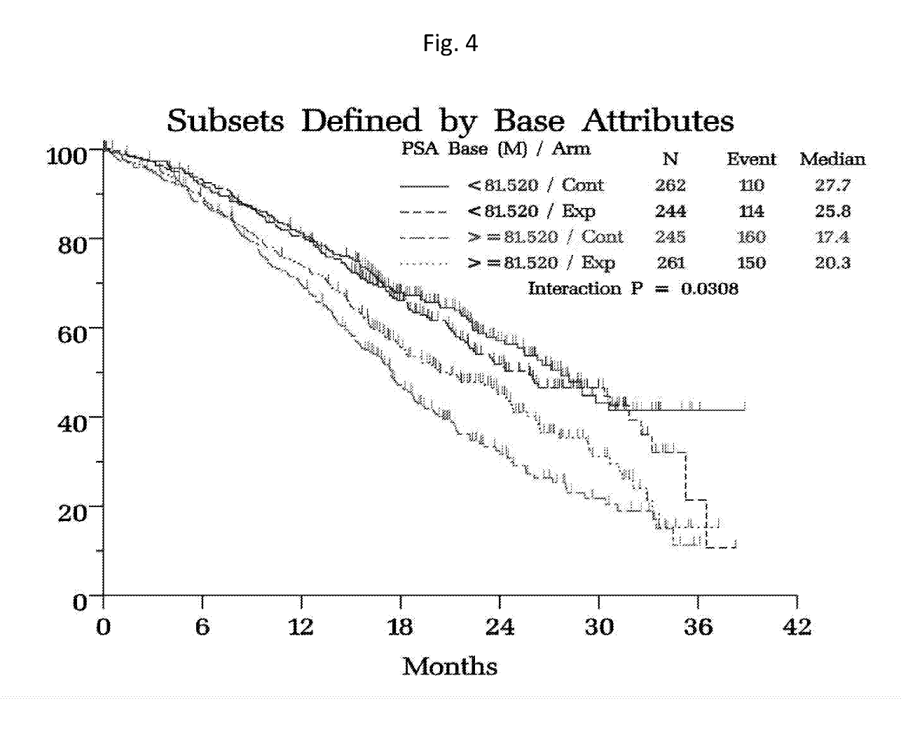

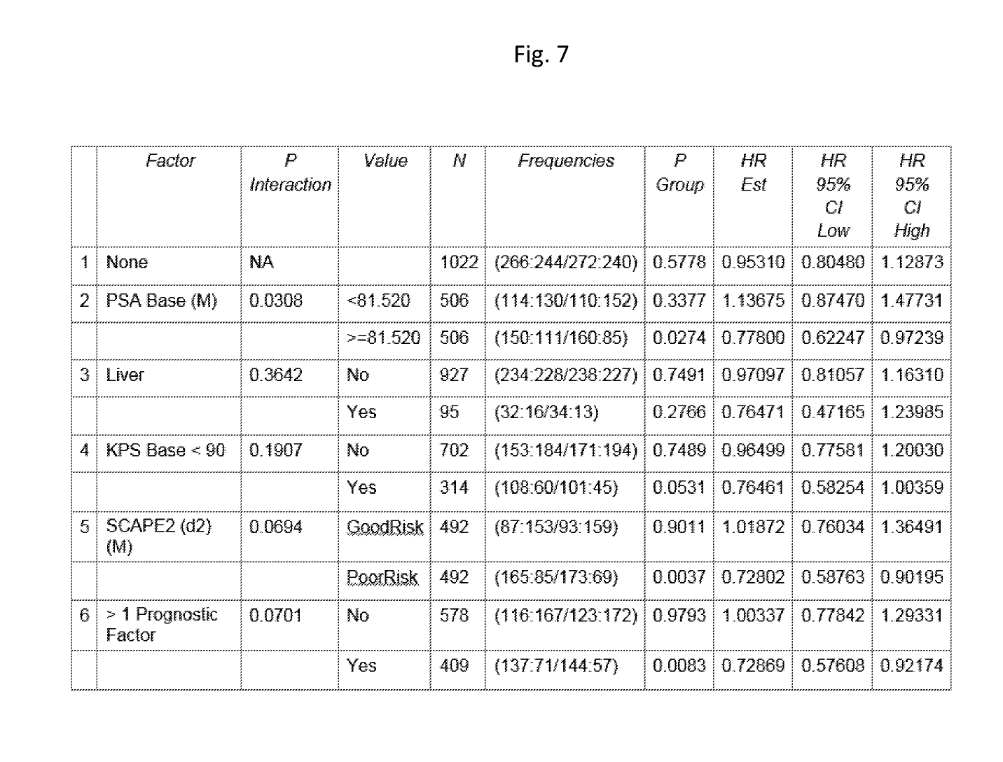

FIG. 7 is a table illustrating the results from this “SCAPE2 (d2)” Systematic Patient Selection Tool. The overall median of SCAPE2 (d2) for all SYNERGY patients was used retroactively to partition SYNERGY patients into poor and good prognosis subgroups. The Kaplan-Meier estimates in FIG. 7 show a custirsen benefit among poor prognosis patients identified using the SCAPE2 (d2) score but not among the good prognosis patients who would not have met the SCAPE2 (d2) criteria. Furthermore, poor prognosis patients identified using SCAPE2 exhibited a larger custirsen effect than what was previously described for the single variable examples as shown in FIG. 2 (presence of Liver metastasis), FIG. 3 (Karnofsky Performance Status) and FIG. 4 (Prostate-Specific Antigen levels). FIG. 7 shows the estimates and confidence intervals as well as model-based P values. The estimated Hazard Ratio (HR) (experimental arm over control arm hazard rates), designated “HR est” is lower for “Poor Risk” patients identified by SCAPE2 or SCAPE2 (d2) than it is for the single example variables.

The simplified SCAPE2 (1) closely matches prognostic performance of the statistical model score, (2) identifies a sufficient proportion of poor prognosis patients, and (3) is easy to implement.

### Example 4

### Study from which Confirmatory Data Derived

The Affinity Trial was a Randomized Phase 3 Study Comparing Cabazitaxel/Prednisone in Combination with Custirsen (OGX-011) to Cabazitaxel/Prednisone for Second-Line Chemotherapy in Men with Metastatic Castrate Resistant Prostate Cancer. The study population were men with metastatic castrate resistant prostate cancer (CRPC) who had already received a docetaxel-containing regimen as first-line chemotherapy and who need second-line chemotherapy due to disease progression. Previous options for second-line chemotherapy included docetaxel retreatment, mitoxantrone, or other chemotherapies without proven clinical benefit. For example, a past Phase 3 second-line chemotherapy trial by another group (the TROPIC trial) showed a survival advantage for cabazitaxel, a semi-synthetic taxane selected to overcome the emergence of taxane resistance, when compared to mitoxantrone.

There were two main objectives for this study. The first was to ascertain whether the survival time distribution for patients randomized to the investigational arm is consistent with longer survival as compared to patients randomized to the control arm for (1) all randomized patients and (2) patients identified as having poor prognosis. The second objective was to compare the arms with respect to the proportion of patients having a milestone Day 140 of being alive and disease progression free.

This was a randomized, open-label, multicenter, international trial. Treatment consisted of cabazitaxel/prednisone/custirsen or just cabazitaxel/prednisone. To be eligible, patients must have had documented progression of prostate cancer after prior first-line docetaxel treatment and meet other inclusion criteria.

Patients were not eligible if they have received chemotherapy for prostate cancer beyond the first-line docetaxel-containing regimen, or if they brain metastases past and/or present, current symptomatic cord compression requiring therapy, active second malignancy, or uncontrolled medical conditions/illnesses that would preclude protocol therapy.

A total of approximately 630 patients were randomized, providing about 315 patients per arm. Patients will be randomly assigned with equal probability to the two arms. Patients received cabazitaxel/prednisone on a 3-week cycle either alone (Arm B) or with weekly custirsen infusions (Arm A) until completion of 10 cycles, disease progression, unacceptable toxicity, or until other withdrawal criteria were met. The date of progression was documented. Patients were and will be followed until death.

Study Agent, Dose and Mode of Administration:

Dose and Mode of Administration:

Custirsen sodium: 640 mg in 250 mL D5W or normal saline IV over 2 hours, given as three IV doses during the loading dose period and weekly thereafter;

Chemotherapy, Dose and Mode of Administration: Chemotherapy: Cabazitaxel and prednisone Dose and Mode of Administration: Cabazitaxel: 25 mg/m2 IV per package insert; and

Prednisone: 10 mg per day.

Duration of Treatment: Arm A patients had a 5- to 9-day loading dose period prior to Day 1 of Cycle 1.

Patients randomized to both study arms (A and B) had 21-day cycles of study treatment until completion of 10 cycles or until disease progression, unacceptable toxicity, or other specific criteria for withdrawal as defined in Section 5.4 of the protocol.

Criteria for Evaluation:

Primary Efficacy Endpoint: Survival time was assessed for each patient from the date of randomization to the date of death from any cause. Secondary Efficacy Endpoint: The status of each patient at approximately Day 140 (window of Day 125-155) post-randomization was recorded as “Alive Without Event” or “Not” (binary outcome) in order to compute the arm-specific proportion of patients who are Alive Without Event at approximately Day 140. This binary endpoint was called the milestone Day 140 status. Patients without an event prior to Day 140 post-randomization had a thorough disease assessment at Day 140 (window of Day 125-155) in order to establish the milestone Day 140 status.

Statistical Methods: The two co-primary efficacy analyses will compare the survival time distributions of patients receiving cabazitaxel and prednisone in combination with weekly custirsen (Arm A) to patients receiving cabazitaxel and prednisone (Arm B) for (1) all randomized patients and (2) poor prognosis patients. The overall type I error probability (alpha) is specified to be one-sided 0.025 and power is specified to be 85% for each objective. The overall alpha is controlled conservatively over these two objectives by sharing alpha; the alpha for objective 1 (all randomized patients) is 0.010 and the alpha for objective 2 (poor prognosis patients) is 0.015. The final analysis for all randomized patients was based on 547 deaths. The final analysis for the poor prognosis subpopulation was 299 deaths. The accrual is specified at 630 patients to be randomized.

The confirmation from the clinical trial that patients with certain disease characteristics are favourably represented in the group experiencing longer survival represents a significant breakthrough in the treatment of mCRPC in patients who have poor prognosis for survival outcome and are receiving second-line chemotherapy for disease progression.

### Example 5

### Computer Aided System Running Method of the Invention

A computer of any size, including a hand-held pocket computer such as a smart phone, is programmed to include the poor prognostic criteria of the invention. A physician or self analysing patient can enter in his values for Karnofsky Performance Status or the ECOG, whether liver metastases are present, and the level of LDH, PSA and Hb in patient's blood. The program emits a message that the patient is suited to improved survival if a clusterin-inhibiting agent such as custirsen sodium is added to the patient's therapy. The program registers the result either on the screen of the computer or emits a sound or voice recording “yes” or “no”. The system is used in the clinic or as an information source for patients.

Thus, in this aspect of the invention, a system is provided comprising a user interface, a memory and a processor.

The user interface receives patient values for a plurality of morbidity factors and communicates a result to a user. The user interface can be in any format, including for example a keyboard, a touch screen, or a voice input/annunciator system, or some combination thereof. Information may also be communicated in digital form to and/or from the user interface.

The memory stores predetermined values for a plurality of morbidity factors as follows:

| Morbidity Factor | Predetermined value |
|---|---|
| Karnofsky Performance Status | ≦80 |
| Liver metastasis | ≧1 |
| LDH (dichotomized) - | LDH ≧360 IU/L |
| PSA (dichotomized) - | ≧150 ng/mL |
| Hemoglobin (dichotomized) | <120 g/L |

The memory may also store received information about patient specific values, although this storage is desirably only of sufficient duration for the performance of operations by the processor.

The processor is configured to compare the values of the received patient morbidity factors to the corresponding predetermined values and provide a first output to a user if two or more of the morbidity factor values conform to the corresponding predetermined value. This first output indicates that the patient is susceptible to prolongation of survival by adding treatment with a clusterin-inhibiting agent. The processor may optionally provide a second output, different from the first output, to the user if two or more of the morbidity factors do not conform to the corresponding predetermined values, thus indicating that treatment with a clusterin-inhibiting agent is not likely to provide additional therapeutic benefit.

### REFERENCES

1. He L R, L. M., Li B K, et al., Clusterin as a predictor for chemoradiotherapy sensitivity and patient survival in esophageal squamous cell carcinoma. Cancer Sci 2009. 100: p. 2354-60.
2. Miyake H, Y. K., Muramaki M, Kurahashi T, Gleave M, Hara I., Enhanced expression of the secreted form of clusterin following neoadjuvant hormonal therapy as a prognostic predictor in patients undergoing radical prostatectomy for prostate cancer. Oncol Rep, 2005(14): p. 1371-5.
3. Pins M R, F. J., Korley F, et al., Clusterin as a possible predictor for biochemical recurrence of prostate cancer following radical prostatectomy with intermediate Gleason scores: a preliminary report. Prostate Cancer Prostatic Dis 2004. 7: p. 243-8.
4. Zhang S, Z. D., Zhu Y, Guo H, Zhao X, Sun B., Clusterin expression and univariate analysis of overall survival in human breast cancer. Technol Cancer Res Treat, 2006. 5: p. 573-8.
5. Kruger S, M. A., Kausch I, Feller A C., Value of clusterin immunoreactivity as a predictive factor in muscle-invasive urothelial bladder carcinoma. Urology, 2006. 67: p. 105-9.
6. Watari H, O. Y., Hassan M K, Xiong Y, Tanaka S, Sakuragi N., Clusterin expression predicts survival of invasive cervical cancer patients treated with radical hysterectomy and systematic lymphadenectomy. Gynecol Oncol, 2008. 108: p. 527-32.
7. James N D, S. M., Clarke N W, et al., Survival with Newly Diagnosed Metastasic Prostate Cancer in the “Docetaxel Era”: Data from 917 Patients in the Control Arm of the STAMPEDE trial (MRC PRO8,CRUK/06/019). Eur Urol, 2015(67): p. 1028-1038.
8. Chi K N, G. M., Antisense approaches in prostate cancer. Expert Opin Biol Ther, 2004. 4: p. 927-36.
9. Gleave M, N. C., Chi K., Antisense targets to enhance hormone and cytotoxic therapies in advanced prostate cancer. Curr Drug Targets, 2003. 4: p. 209-21.
10. Henry S, S. K., Brooks D, Monteith D, Conklin B, Bennett C F., Chemically modified oligonucleotides exhibit decreased immune stimulation in mice. J Pharmacol Exp Ther, 2000. 292: p. 468-79.
11. Henry S P, G. R., Yu R, Levin A A., Drug properties of second-generation antisense oligonucleotides: how do they measure up to their predecessors?. Curr Opin Investig Drugs 2001. 2: p. 1444-9.
12. Zellweger, T., et al., Antitumor activity of antisense clusterin oligonucleotides is improved in vitro and in vivo by incorporation of 2′-O-(2-methoxy)ethyl chemistry. The Journal of pharmacology and experimental therapeutics, 2001. 298(3): p. 934-40.
13. Gleave M, M. H., Zangemeister-Wittke U, Jansen B., Antisense therapy: current status in prostate cancer and other malignancies. Cancer Metastasis Rev 2002, 2002. 21: p. 79-92.
14. Chi, K. S. J. H., E. Yu, D. Tu, B. Eigl, I. Tannock, F. Saad, S. North, J. Powers, E. Eisenhauer, E., Mature results of a randomized phase II study of OGX-011 in combination with docetaxel/prednisone versus docetaxel/prednisone in patients with metastatic castration resistant prostate cancer. J Clin Oncol, 2009. 27(15s): p. #5012.
15. Oken, M. M., et al., Toxicity and response criteria of the Eastern Cooperative Oncology Group. Am J Clin Oncol, 1982. 5(6): p. 649-55.
16. Szklaruk, S. M. a. J., Magnetic resonance imaging: Review of imaging techniques and overview of liver imaging. World J Radiol, 2010. 2(8): p. 309-322.
17. Halabi, S., Chen-Yen Lin, Eric J. Small, Andrew J. Armstrong, Ellen B. Kaplan, Daniel Petrylak, Cora N. Sternberg, Liji Shen, Stephane Oudard, Johann de Bono, and Oliver Sartor, Prognostic Model Predicting Metastatic Castration-Resistant Prostate Cancer Survival in Men Treated With Second-Line Chemotherapy. J National Cancer Inst, 2013. 105(22): p. 1729-173.
18. Halabi, S., Chen-Yen Lin, W. Kevin Kelly, Karim S. Fizazi, Judd W. Moul, Ellen B. Kaplan, Michael J. Morris, and Eric J. Small, Updated Prognostic Model for Predicting Overall Survival in First-Line Chemotherapy for Patients With Metastatic Castration-Resistant Prostate Cancer J Clin Oncol, 2014: p. 671-677.

1. A method for determining if a prostate cancer-afflicted patient is susceptible to survival prolongation through treatment with a clusterin-inhibiting agent in combination with a chemotherapy agent, the method comprising the steps of:
(a) obtaining a plurality of test results for a patient, said test results being selected from the group consisting of:
(i) a Performance Scale result,
(ii) the presence of metastatic lesions on the patient's liver,
(iii) blood prostate specific antigen (PSA) level,
(iv) blood lactose dehydrogenase (LDH) level, and
(v) hemoglobin (Hb) levels;
(b) comparing each of the obtained test results to a predetermined value for the test result indicative of a poor prognosis; and
(c) if any two of the resulting five measurements indicate poor prognosis in a cancer patient concluding that said patient is susceptible to survival prolongation through treatment with a clusterin-inhibiting agent in combination with a chemotherapy agent.

2. The method according to claim 1, wherein the Performance Scale result is a Karnofsky Performance Status and the predetermined value is less than or equal to 80, or an ECOG and the predetermined value is greater than 1;

3. The method according to claim 1 wherein the predetermined value for PSA level is greater than or equal to 150 ng/L.

4. The method according to claim 1, wherein the predetermined level for Hb is less than 120 g/L.

5. The method of claim 1, wherein the predetermined level of LDH is greater than or equal to 360 UNITS/L.

6. A method of treating metastatic castrate resistant prostate cancer in a patient needing such treatment, comprising the steps of
determining if the patient is susceptible to survival prolongation through treatment with a clusterin-inhibiting agent in combination with a chemotherapy agent by performing the method of claim 1, and
if the patient is determined to be susceptible to survival prolongation, including a clusterin-inhibiting agent in the standard treatment for the patient.

7. The method of claim 6, wherein the clusterin-inhibiting agent is an antisense oligonucleotide.

8. The method of claim 7, wherein the antisense oligonucleotide is Custirsen.

9. The method of claim 8, wherein the standard treatment includes treatment with a taxane.

10. The method of claim 9, wherein the taxane is docetaxel.

11. A system comprising:
a user interface for receiving patient values for a plurality of morbidity factors and communicating a result to a user,
a memory, said memory storing predetermined values for a plurality of morbidity factors as follows:

| Morbidity Factor | Predetermined value |
|---|---|
| Karnofsky Performance Status | ≦80 |
| Liver metastasis | ≧1 |
| LDH (dichotomized) - | LDH ≧360 IU/L |
| PSA (dichotomized) - | ≧150 ng/mL |
| Hemoglobin (dichotomized) | <120 g/L |

and,
a processor configured to compare the values of the received patient morbidity factors to the corresponding predetermined values and provide a first output to a user if two or more of the morbidity factor values conform to the corresponding predetermined value.

12. The system of claim 11, wherein the processor provides a second output, different from the first output, to the user if two or more of the morbidity factors do not conform to the corresponding predetermined values.
